(19) United States
(12) Patent Application Publication
(10) Pub. No.: US 2017/0020605 A1
(43) Pub. Date: Jan. 26, 2017

(54) Hyperthermia Electromagnetic Energy Applicator Housing and Hyperthermia Patient Support System

(71) Applicant: Pyrexar Medical Inc., Salt Lake City, UT (US)

(72) Inventors: Jason Ellsworth, Farmington, UT (US); Paul F. Turner, Bountiful, UT (US)

(21) Appl. No.: 15/190,098

(22) Filed: Jun. 22, 2016

Related U.S. Application Data
(60) Provisional application No. 62/183,183, filed on Jun. 22, 2015.

(51) Int. Cl. A61B 18/18; A61N 1/40

(52) U.S. Cl. CPC A61B 18/18; A61N 1/403; A61B 2018/00172

(57) ABSTRACT

EMR applicators of an EMR applicator array are provided in an openable applicator housing to allow easy patient entrance to and exit therefrom so that the portion of the patient's body that contains the tissue to be treated can be positioned directly into the applicator housing without the applicator housing having to be moved along the patient's body. This can eliminate or reduce the need for full body supporting structure as part of the applicator housing. The applicator housing provides support for the portion of the body positioned therein, and support pads or pillows, separate from the applicator housing, can replace the need for a full body support as part of the applicator housing. The housing can reduce the size and complication of prior art applicator housings, and can be placed along with the patient, on a patient support surface, such as in a standard MRI system.

### PRIORITY CLAIM

Priority is claimed to copending U.S. Provisional Patent Application Ser. No. 62/183,183 filed Jun. 22, 2015, which is hereby incorporated herein by reference in its entirety.

### BACKGROUND OF THE INVENTION

Field

The present invention relates generally to systems and apparatus for irradiating patients with electromagnetic radiation, and more specifically to systems having annular-type or sectored arrays of applicators and associated control systems for controlling application of radiation to patients through phased array power steering, wherein the patient is supported within the annular-type or sectored array of applicators wherein the applicators surround a portion of the patient.

State of the Art

Hyperthermia, the generation of artificially elevated body temperatures, has recently been given serious scientific consideration as an alternative cancer treatment. Much research has been conducted into the effectiveness of hyperthermia alone or in combination with other treatment methods. Hyperthermia techniques appear to have the potential for being extremely effective in the treatment of many or most types of human cancers, without the often severely adverse side effects associated with current cancer treatments such as chemotherapy or radiation. Hyperthermia is sometimes called thermal therapy indicating the raising of the temperature of a region of the body.

Hyperthermia is generally provided by temperatures over 40 degrees C. (104 degrees F.). Hyperthermia has historically included temperatures well above 60 degrees C., but in recent years has generally been considered to include temperatures as high as 45 degrees C. (113 degrees F.). Currently treatments using temperatures well above 45 degree C. to directly kill or ablate tissue, such as cancer tissue, is usually referred to as ablation rather than hyperthermia, but the term hyperthermia as used herein can include either type of heating and treatment. At treatment temperatures above the approximate 45 degrees C. (113 degrees F.), thermal damage to most types of normal cells is routinely observed if the time duration exceeds 30 to 60 minutes; thus, great care must be taken not to exceed these temperatures in healthy tissue for a prolonged period of time. Exposure duration at any elevated temperature is an important factor in establishing the extent of thermal damage to healthy tissue. If large or critical regions of the human body are heated into, or above, the 45 degree C. temperature range for even relatively short times, normal tissue injury may be expected to result. With any such heat treatment, the intent is to get as much of the cancerous tissue as possible above the 40 degree C. temperature, without heating the normal tissue surrounding the cancerous tissue to temperatures which will kill or damage the normal tissue. Therefore, it is desirable to be able to selectively heat cancerous tissue in a mass of normal tissue, such as in a human body, to desired increased temperatures without heating the normal tissue.

One way to heat tissue is to apply electromagnetic radiation (EMR) to such tissue. One currently used method for applying electromagnetic radiation (EMR) to selected targets, such as living bodies and biological tissue, and for controlling the position of a region of heating within the target is through phased array power steering. Systems that use phased array power steering provide a plurality of electromagnetic energy applicators positioned around a portion of a living body or tissue mass to be treated wherein the power and phase of the electromagnetic energy radiated by each electromagnetic energy applicator can be controlled to control the size and location of an area of heating within the living body or tissue mass. Generally, the plurality of electromagnetic energy applicators will be positioned to surround the living body or tissue mass to be treated. With such systems, the array of electromagnetic energy applicators are generally provided in a housing wherein the electromagnetic energy applicators form at least one ring within the housing around an opening in the housing adapted to receive the living body or tissue mass therein. When the living body or tissue mass is a human patient, the patient is supported with the portion of the patient containing the tissue to be treated, such as the pelvis, abdomen, or thorax of the patient, within the opening of the housing so that the electromagnetic energy applicators in the housing substantially encircle the portion of the patient containing the tissue to be treated. With such arrangement, the electromagnetic energy applicators are spaced around the patient a distance away from the patient. The housing generally includes an inflatable bolus around the opening which can be filled with a dielectric fluid having an impedance approximately equivalent to an applicator impedance at the frequency of the EMR energy radiation being used in the system to fill the space between the electromagnetic energy applicators and the surface (skin) of the portion of the patient received in the opening. The dielectric fluid will generally be deionized water. Examples of such prior art systems are shown and described in U.S. Pat. Nos. 4,672,980; 5,097,844; 7,565,207; and 8,170,643, all of which are incorporated herein by reference. A commercial system is available as the BSD-2000 system from Pyrexar Medical Inc. in Salt Lake City, Utah.

As indicated, prior art systems, such as the BSD-2000, provide a housing which includes an opening through which the patient is inserted and the portion of the patient having the tissue to be treated is in the opening. With the patient positioned in the opening in the housing, a bolus, included as part of the housing, is filled with deionized water to contact the portion of the patient in the opening in the housing and provide the deionized water between the electromagnetic energy applicators in the housing and the patient's body portion positioned in the opening in the housing. In order to accurately position the patient in the opening in the housing, a fabric sling patient support is provided. FIGS. 1 and 2 show BSD-2000 systems with a patient 10 positioned in opening 12 in applicator housing 14, which includes a cylindrical applicator holder 16 with bolus 18 inflated with deionized water around the patient. As used herein, cylindrical does not mean circular in cross section as the illustrated cylindrical applicator holder 16 is shown as being substantially elliptical in cross section, but merely means that it surrounds the patient. The electromagnetic energy applicators are secured to and spaced around the inside of the cylindrical applicator holder 16, and are inside the bolus 18. The patient 10 is supported on a fabric sling 20 supported along opposite sides by side support tubes 22. The ends of the respective side support tubes 22 are connected by end support tubes 24. Brackets 26 connect side support tubes 22 through end support tubes 24 to vertical corner supports 28. In the embodiment of FIG. 1, the vertical corner supports 28 extend from system base 30. Vertical corner supports 28, and brackets 26 attached thereto, are height adjustable to enable the fabric sling 20 to be height adjustable for adjusting the height and inclination of the sling 20 and patient 10 thereon in the applicator housing 14. The applicator housing 14 is mounted on track 32 for longitudinal movement on system base 30 with respect to sling 20 and patient 10 thereon to align the applicator housing 14 with the portion of the patient's body containing the tissue to be treated. With this arrangement, the applicator housing 14 can be moved along the track 32 to an end of the sling 20 to allow easy access of the patient to or exit of the patient from sling 20. Once the patient is on sling 20, applicator housing 14 is moved along track 32 and sling 20 to the desired position along the patient's body. When positioned as desired along the patient's body, such as around the patient's abdomen as shown in FIG. 1, the height of the patient's body in applicator opening 12 with respect to the cylindrical applicator holder 16 in applicator housing 14 can be adjusted by adjusting corner supports 28. Vertical corner supports 28 are generally controlled and adjusted by hydraulic lifts in system base 30. When in desired position, bolus 18 is filled with deionized water to contact the patient above the sling 20 and to contact the bottom of the sling 20 which serves as the contact of the bolus with the portion of the patient resting on sling 20, the bolus also forming itself around the edges of the sling and the sling side supports 22. This arrangement works satisfactorily for a system such as shown in FIG. 1 where the system performs its treatment standing as shown in FIG. 1. The height and tilt adjustment of the patient in the applicator housing as provided by the fabric sling 20 and adjustable corner supports 28 is important because hyperthermia treatments are usually repeatedly performed at time intervals, such as weekly, for a predetermined plurality of treatments. Each treatment should be the same as the previous treatments which means that the patient has to be in the same position in the applicator housing for each treatment. The adjustability is necessary so the position of the patient for each treatment can be adjusted to be substantially the same as the previous treatments.

As indicated above, it is important when heating tissue to be heat treated, that the surrounding normal tissue is not heated to an extent to damage the normal tissue. Therefore, it is important to monitor the temperature of at least the normal tissue at or near the outer edge of the tissue being heated. Systems such as shown in FIG. 1 include various temperature sensing apparatus and methods to monitor various tissue temperatures. In one embodiment of the prior art BSD-2000 system, it has been found that temperature of tissue being treated and surrounding tissue can be accurately monitored by a magnetic resonance imaging (MRI) system, and that the hyperthermia treatment can take place in the MRI system which then monitors the temperature of the tissue being treated and surrounding tissue during the treatment, see referenced U.S. Pat. No. 8,170,643. An embodiment of the BSD-2000 system for use with a prior art MRI system is shown in FIG. 2 where the MRI system is indicated generally by reference number 40, with the opening therein into which the patient is positioned is indicated by reference number 42. The various commercially available MRI systems generally have a support base extending from the MRI opening and a patient table structure that moves between the support base and the inside of the MRI opening to transport the patient, on a thin pad on the table, into the MRI system for diagnostic imaging. However, the normal MRI patient table structure is not configured to accept a hyperthermia support system. For a hyperthermia treatment in an MRI system, the patient, as positioned in applicator housing 14, has to be moved into and positioned in the MRI system. This requires a complete patient hyperthermia system that can be inserted into the MRI opening 42, such system including both the applicator housing 14 which applies the hyperthermia treatment and the patient support system (fabric sling 20) that supports, positions, and holds the patient in the appropriate position in the applicator housing 14. For an MRI hyperthermia system, a special narrow height system base 44, FIG. 2, which includes the applicator housing mounting track 32 and the vertical corner supports 28 for fabric sling 20, along with the hydraulic lift system for the vertical corner supports, has to be constructed along with a separate auxiliary supporting base 46 which supports the narrow height system base 44 at the appropriate height so it can be slide off the auxiliary supporting base 46 into the MRI opening 42. Further, since the various MRI system suppliers and the different MRI system models all have different support structures and patient transfer mechanisms, the hyperthermia systems for insertion into the MRI systems generally need small variations to fit different models of MRI systems. In addition, it has been found that even with the special narrow height system base 44, the patient and applicator array are at a very high position when that structure is placed on a standard support structure of an MRI system which often places the patient and reference devices used to compensate for magnetic field drift and non-uniformity out of the field of view of the MRI system.

There is a need for EMR applicator apparatus as part of a hyperthermia system that, together with a patient, can be placed on top of standard MRI system patient support surfaces so the patient in a hyperthermia applicator housing can be inserted into an MRI opening.

### SUMMARY OF THE INVENTION

According to the invention, it has been found that the EMR applicators of an EMR applicator array can be provided in an openable applicator housing to allow easy patient entrance to and exit therefrom so that the portion of the patient's body that contains the tissue to be treated can be positioned directly into the applicator housing without the applicator housing having to be moved along the patient's body, with the patient's body in an opening in the housing, to the portion of the patient's body that contains the tissue to be treated. This can eliminate or reduce the need for full body supporting structure as part of the applicator housing. The applicator housing can provide support for the portion of the body positioned therein, and support pads or pillows, separate from the applicator housing, can replace the need for a full body support, such as a full body length fabric sling, as part of the applicator housing. Such an applicator housing can substantially reduce the size and complication of prior art applicator housings, and can provide an applicator housing that can be placed along with the patient, on a patient support surface, such as the usual patient support surface of a standard MRI system.

In one embodiment of the invention, the applicator housing is in the form of a clam shell structure which is openable to allow a patient to place the portion of the patient's body that contains the tissue to be treated directly into the applicator housing. The usual patient support sling supporting the patient's body extending through the applicator housing and from the ends of the applicator housing is replaced by simple foam padding on a patient support surface at both ends of the applicator. These pads can then support the patient to a desired position, such as the approximate center of a dipole antenna array, in the applicator housing. The clam shell design allows the patient to first sit at one end of the applicator housing on a pad and then by rotating ninety degrees and lying down, to be properly positioned in the applicator housing. The patient is supported by pads adjacent both ends of the applicator housing so the portion of the body extending through the applicator housing will generally be suspended and supported in the housing and not need direct support when entering and exiting the housing. A bolus in the lower part of the housing can be already filled with deionized water to provide a water bed type of support surface for the patient as the patient lies down, or a short fabric sling secured in the applicator housing can provide a support surface for the patient as the patient lies down. In either case, once the patient lies down in the applicator housing, the clam shell structure can be closed over the portion of the patient's body in the applicator housing and the upper section of the water bolus, and the lower portion, if not yet filled or completely filled, filled before proceeding to the treatment. By using separate support pads and the clam shell design, the fit of the applicator housing structures needed to provide the hyperthermia treatments can be adapted to a size and shape to fit onto standard patient support structures, such as a standard patient support structure used with MRI systems.

### THE DRAWINGS

Additional features and advantages of the invention will be apparent from the detailed description which follows, taken in conjunction with the accompanying drawings, which together illustrate, by way of example, features of the invention; and, wherein:

FIG. 1 is a perspective view of a free standing prior art BSD-2000 hyperthermia system;

FIG. 2 is a perspective view of a prior art modified BSD-2000 hyperthermia system adapted for use in combination with an MRI system;

FIG. 3 is a pictorial view of an applicator housing of the invention with the applicator housing in open condition;

FIG. 4 is a pictorial view of the applicator housing of FIG. 3 with the applicator housing in closed condition;

FIG. 5 is an end view of the applicator housing of FIG. 4 in closed condition;

FIG. 6 is a vertical section through the applicator housing of FIG. 4 in closed condition;

FIG. 7 is a vertical section through the applicator housing similar to that of FIG. 6, showing a different embodiment;

FIG. 8 is an vertical section through the applicator housing similar to that of FIG. 6, showing a still different embodiment;

FIG. 9 is a pictorial view of the applicator housing of FIGS. 1-6 in opening condition resting on a patient loading base of an MRI system along with a plurality of patient support pads also resting on the patient loading base adjacent the ends of the applicator housing;

FIG. 10 is a pictorial view similar to that of FIG. 9, but showing the applicator housing in closed condition;

FIG. 11 is a pictorial view similar to that of FIG. 9, but showing a patient positioned in the open applicator housing and also supported by the adjacent pads;

FIG. 12 is a pictorial view similar to that of FIG. 11, but showing the applicator housing in closed condition ready to be moved into the MRI system.

FIG. 13 is a pictorial view similar to that of FIG. 2 showing a different embodiment of an applicator housing of the invention with the applicator housing in open condition;

FIG. 14 is a vertical section through the applicator housing of FIG. 13, but with the housing in closed condition; and

FIG. 15 is a pictorial view of the applicator housing of FIG. 13, but with the housing in closed condition, with the cover removed, and taken at a different angle.

### DETAILED DESCRIPTION OF THE ILLUSTRATED EMBODIMENTS

The present invention provides an EMR applicator array housing that is openable to allow easy patient entrance to and exit therefrom so that the portion of the patient's body that contains the tissue to be treated can be positioned directly into the applicator housing. The applicator housing can provide support for the portion of the body positioned therein, and support pads or pillows, separate from the applicator housing, can support the portions of the patient body outside of the applicator housing.

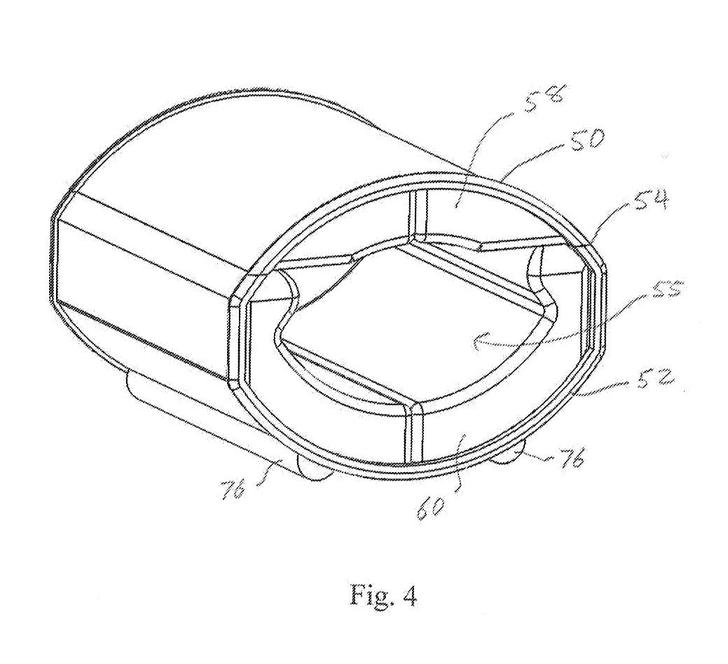

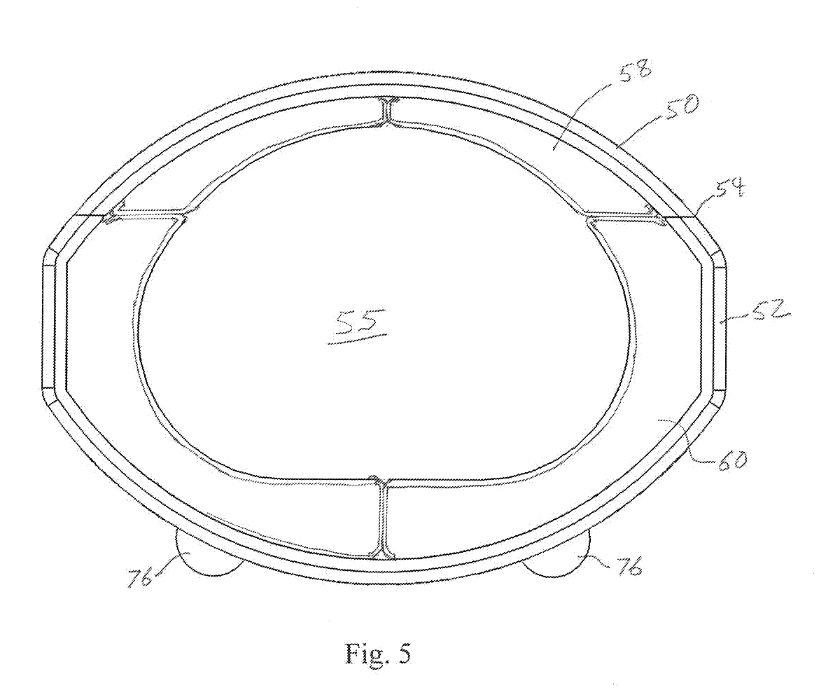

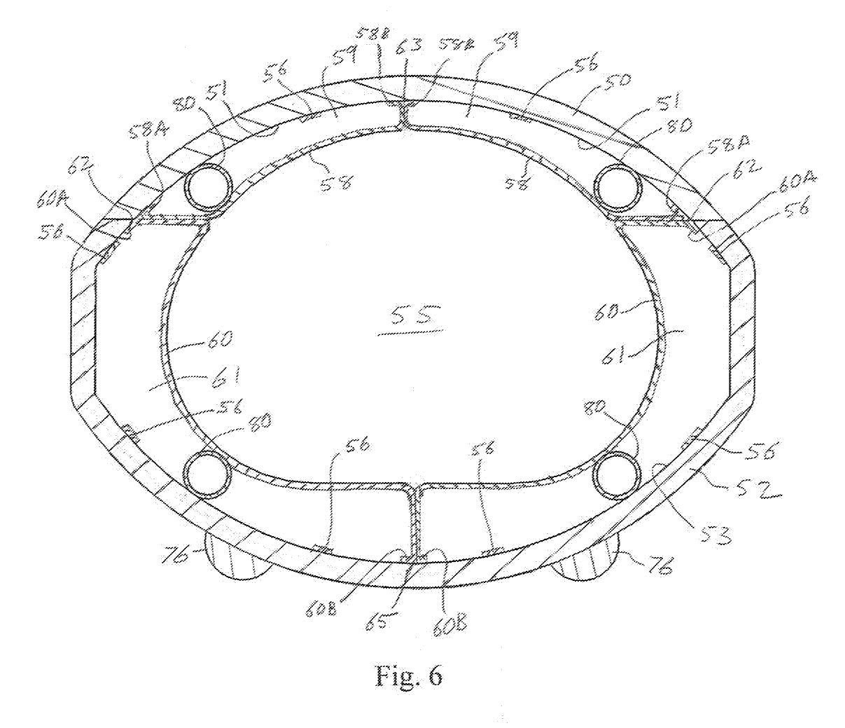

An example embodiment of the openable EMR applicator housing of the invention is illustrated as of clamshell configuration. An upper housing shell 50, which forms an inner upper concave surface 51, FIGS. 6-8, is hingedly secured to a lower housing shell 52, which forms an inner lower concave surface 53, such as by a hinge connector along the connecting edges 54. Various types of hinge connectors can be used. The shells can be moved between an open condition as shown by FIG. 3 and a closed condition shown by FIGS. 4, 5, and 6. A connector, not shown, is provided to latch the shell together in closed condition. Again, various types of connectors can be used. The upper and lower shells are made of dielectric material such as a plastic material. When in closed condition, the inner upper concave surface 51 faces the inner lower concave surface 53 to form a cylindrical shell with an opening 55 extending from end to end therethrough. EMR applicators, shown schematically as boxes 56, FIGS. 6-8, and shown larger than actual scale size, are substantially evenly and/or symmetrically spaced and attached around the inside concave surfaces of the shells. Such EMR applicators can be in the form of dipole antennas formed on or attached to the inside concave surfaces of the upper and lower shells. Electrical cables, not shown, will connect the EMR applicators to a source of EMR energy outside of the applicator housing. Depending upon the hyperthermia system used, the EMR applicators can form a single ring of applicators around the shell or multiple rings of applicators around the shell. For example, if an applicator array configuration as shown in referenced U.S. Pat. No. 5,097,844 is to be provided to allow three dimensional positional steering and focusing of the heating pattern created by the applicator array, three rings of eight applicators each would be provided. Any desired number of rings and number of applicators per ring can be formed in the housing.

As indicated, when in closed condition, the inner upper concave surface 51 faces the inner lower concave surface 53 to form a cylindrical shell with an opening 55 extending from end to end therethrough. As used herein, cylindrical does not mean circular in cross section as the illustrated cylindrical shell and opening 55 extending therethrough is shown as being substantially elliptical in cross section, but merely means that it surrounds the patient. For treatment, the cylindrical shell is positioned around a portion of a patient's body, such as the patient's trunk or torso, containing the tissue to be treated. The upper portion and the lower portion of the patient's body extend from the ends of the applicator housing. Within the applicator housing, a bolus is provided which is filled with a dielectric fluid, such as deionized water, so that the bolus extends against the patient's body in opening 55 and provides a dielectric fluid in the space between the surface of the patient's body and the inside surface of the applicator housing. With a clamshell configuration with an upper housing shell and a lower housing shell, an upper bolus is provided on the upper shell and a lower bolus is provided on the lower shell.

In the illustrated embodiments of FIGS. 3-12, flexible plastic sheet material is attached around the edges of the shells to form a bolus on each shell. As shown, flexible plastic sheet material 58 is attached to the edges, both the longitudinal and end edges, of top shell 50 to form a top shell bolus having an interior space 59, FIGS. 6-8, and flexible plastic sheet material 60 is attached to the edges, both the longitudinal and end edges of bottom shell 52 to form a bottom shell bolus having an interior space 61. The boluses can be inflated with a dielectric fluid, such as deionized water, to contact a portion of a patient body surface when the patient body portion is positioned in opening 55 created inside the housing when the housing is in closed condition as shown in FIGS. 4-8. The dielectric fluid in the bolus spaces 59 and 61 will fill the area between the EMR applicators 56, FIGS. 6-8, and the outside surface of the body received in the EMR applicator opening when closed, and the flexible material forming the boluses will conform to and abut the outside surface of the body to provide impedance matching between the EMR applicators and body. The bolus spaces will be connected to a source of dielectric fluid so they can be controllably filled and emptied when desired and to the extent desired.

As shown in FIGS. 3-6, the upper bolus is actually formed as two boluses, with the longitudinal edges 58A of two sheets of flexible plastic material 58 secured, such as by gluing, along inside upper concave surface longitudinal edges 62 of such surface and longitudinal edges 58B of the two sheets of flexible plastic material 58 secured, such as by gluing, along a longitudinal strip 63 along inside upper concave surface 51 intermediate the longitudinal edges of inner upper concave surface 51. Similarly, the lower bolus is actually formed as two boluses, with the longitudinal edges 60A of two sheets of flexible plastic material 60 secured, such as by gluing, along inside lower concave surface longitudinal edges 64 of such surface and longitudinal edges 60B of the two sheets of flexible plastic material 60 secured, such as by gluing, along a longitudinal strip 65 along inside lower concave surface 53 intermediate the longitudinal edges of the inner lower concave surface 53. The two upper boluses can be connected so are filled from the same source of dielectric fluid, or can be separately connected to sources of dielectric fluid. One reason to provide the two upper boluses is that two smaller boluses with the intermediate connection to the inner upper concave surface 51 will maintain a more conforming shape to the inner upper concave surface 51 during movement between the open and closed positions, although usually the upper bolus space will be drained prior to opening and closing. Similarly, two lower smaller boluses with the intermediate connection to the inner lower concave surface 53 will maintain a more conforming shape to the inner lower concave surface 53 which may provide better centering of the patient body portion during insertion of the patient body portion onto the lower boluses when the housing is in open condition.

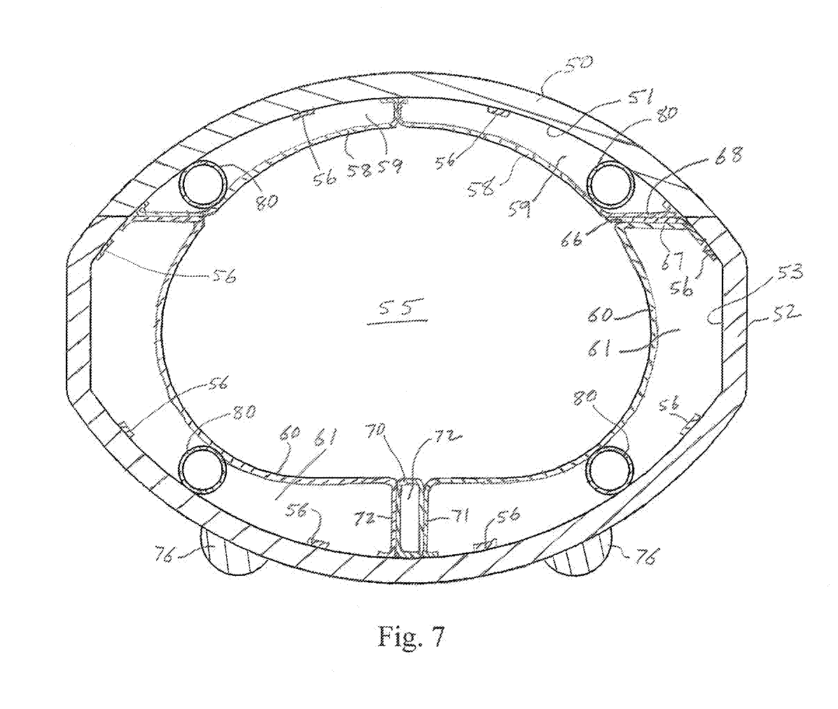

In some embodiments of the invention, it will be advantageous to provide for low dielectric separation zones within the fluid bolus to decrease cross-coupling between applicator array sectors. When higher frequencies of EMR energy are used, there can be standing waves within the fluid bolus that can alter the distribution of the EMR fields and degrade the deep focus of the EMR power. Low dielectric separator sectors along the body length can be used to isolate the EMR fields from adjacent applicator sectors to reduce such cross-coupling between applicators. Such sector dividers can extend from the body surface area to the inner surface of the housing shells. FIG. 7 illustrates two different embodiments of a low dielectric sector separator that could be used between boluses and or bolus section. As shown in FIG. 7, such a low dielectric sector separator can take the form of a strip of low dielectric material, such as a low dielectric plastic or low dielectric rubber 66 secured along the edge 67 of material 60 forming the lower bolus where the edge 67 of the lower bolus meets the edge 68 of the material 58 forming the upper bolus when the edges 67 and 68 of the lower bolus and upper bolus, respectively, come together as the upper housing shell 50 is moved to closed condition. An alternate form of low dielectric sector separator can take the form of a compartment 70 positioned between adjacent edges 71 and 72 of adjacent boulses, such as shown between the two lower boluses in FIG. 7, with the compartment filled with a low dielectric material 72 such as air, foam, or low dielectric rubber or plastic. While two alternate examples of low dielectric sector separators have been illustrated in FIG. 7 between two instances of adjoining bolus edges, either type of low dielectric sector separator, or other low dielectric sector separators, can be used along all adjacent edges separating all boluses of an EMR energy applicator housing of the invention.

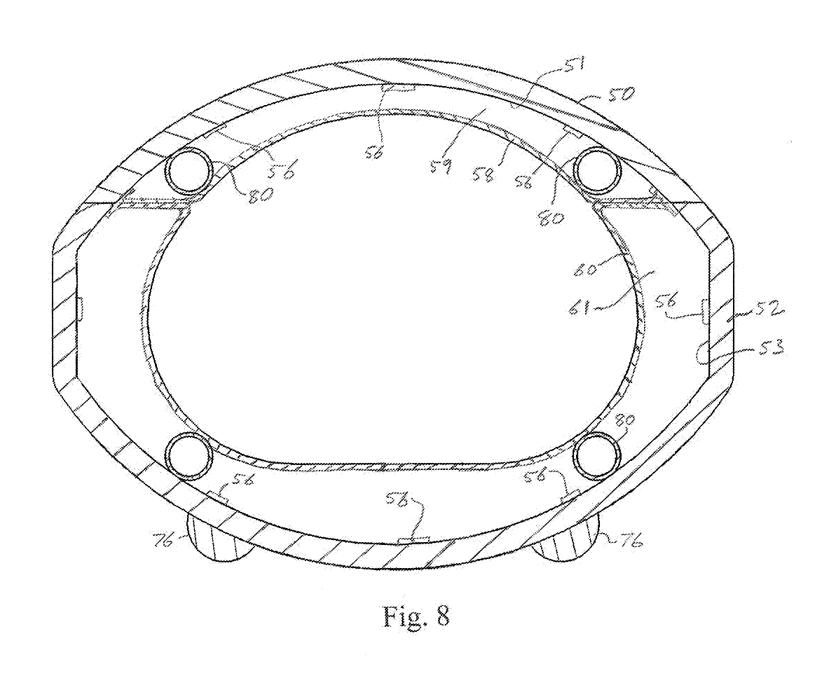

An alternate embodiment of the invention shown in FIG. 8 provides a single upper bolus and a single lower bolus. Such a construction is usually satisfactory. With such two bolus construction, the upper and lower bolus sections can be joined at the hinged area so the two bolus spaces 59 and 61 can be filled with dielectric fluid through the same dielectric fluid inlet and drained through the same dielectric fluid outlet. In such situation, the lower bolus can remain filled or partially filled while the upper bolus can be completely drained since fluid will flow from the upper bolus into the lower bolus during draining. Rather than the two boluses being joined, the two boluses can be separated. Separation allows the pressure applied to the lower bolus to elevate or lower the patient body portion resting on the lower bolus to adjust the vertical position of the patient body portion relative to the applicator array in the housing. To do this the lower bolus is connected to a separate dielectric fluid supply.

Supporting feet 76 can be provided to support the applicator housing on a supporting surface.

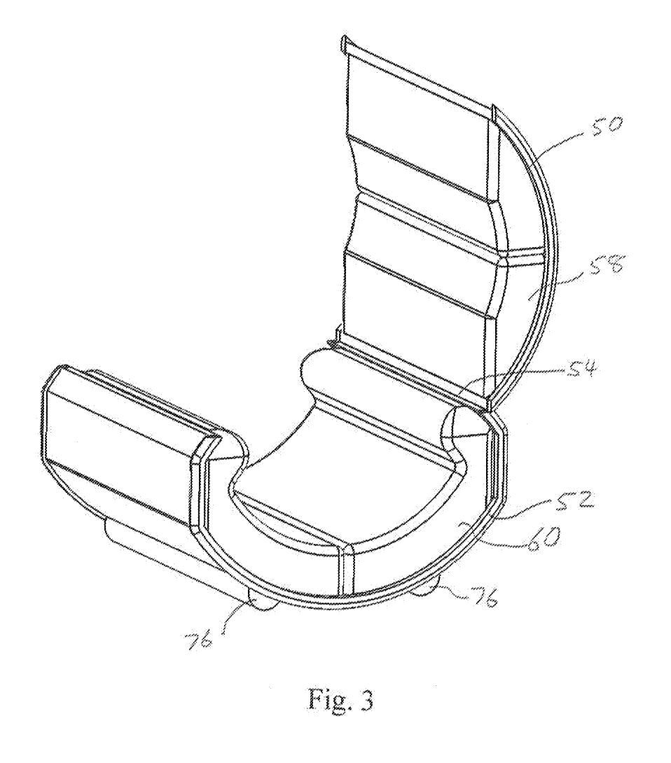

As previously indicated, a hyperthermia system can be combined with an MRI system and the EMR energy applicator along with the patient are positioned in the MRI system so that the hyperthermia treatment takes place in the MRI system where the MRI system is used to monitor temperature of the tissue within the body being treated and the tissue surrounding the tissue being treated. When an EMR energy applicator housing with a bolus as part of the housing is used to provide hyperthermia treatment in an MRI system, it is necessary to compensate the temperature information obtained by the MRI system for non-uniformities and changes of the magnetic field during the treatment. For this purpose, tubular types of structures oriented along the body length are provided in the EMR energy applicator housing to be used as references to allow for such compensation of non-uniformities and changes of the magnetic field during the treatment. These tubular objects are typically filled with a material such as silicone gel which is a material that does not have a temperature variable image, but that does change its image intensity as the magnetic field changes. These tubular types of structures, which will be referred to here as gel tubes, can be positioned within a bolus or outside of a bolus. The use of these gel tubes is part of the current BSD-2000 hyperthermia MRI integrated systems as shown in FIG. 2, but are not visible as they are located inside the bolus of such systems. These gel tubes are also used with the EMR applicator housings of the current invention and are shown within the boluses of the embodiments of FIGS. 3-8 and are shown in section as tubes 80 in the sectional views, FIGS. 6, 7, and 8. Typically there are four such gel tubes which are attached to the inner upper and lower concave surfaces 51 and 53 of the upper and lower housing shells 50 and 51, respectively. These tubes extend into the boluses, but do not extend all the way to the body surface in opening 55 thereby leaving space between the inner sides of the tubes and the inner walls of the boluses so the tubes do not interfere with various sizes of patient bodies received in the bolus opening. Of these four gel tubes 80, two gel tubes are typically displaced vertically above the patient or in the upper vertical portion of the patient's body and two gel tubes are typically at the vertical position that corresponds with the lower portion of the patient's body. These allow reference points to be available to show the non-uniformity of the magnetic field within these zones which will only have differences in their MRI image intensity due to the magnetic field strength in that region. Temperature changes in the body tissues during treatment cause changes in the intensity of the real and imaginary portions of the MR image data as well as differences in the magnetic field. The measurement of the intensity image of these gel tubes allows a software program to modify the MRI generated images to cause the image at each of the gel tubes to be uniform. This is done by a process such as bi-linear interpolation that is also applied to the rest of the image areas of the patient's body and bolus. This process corrects temperature change images that are obtained by subtracting the body image obtained prior to elevating the patient's body temperature from those measured during the heat treatment process. The imaginary portion of the MRI image is normally what is used to measure the temperature changes. This imaginary portion of the imaging data is generated by a shift in the proton resonance frequency and is primarily changed as a result of tissue temperature changes. There are other MRI imaging methods that can also be used, such as a change in the coefficient of diffusion image which is also largely effected by the change in tissue temperature.

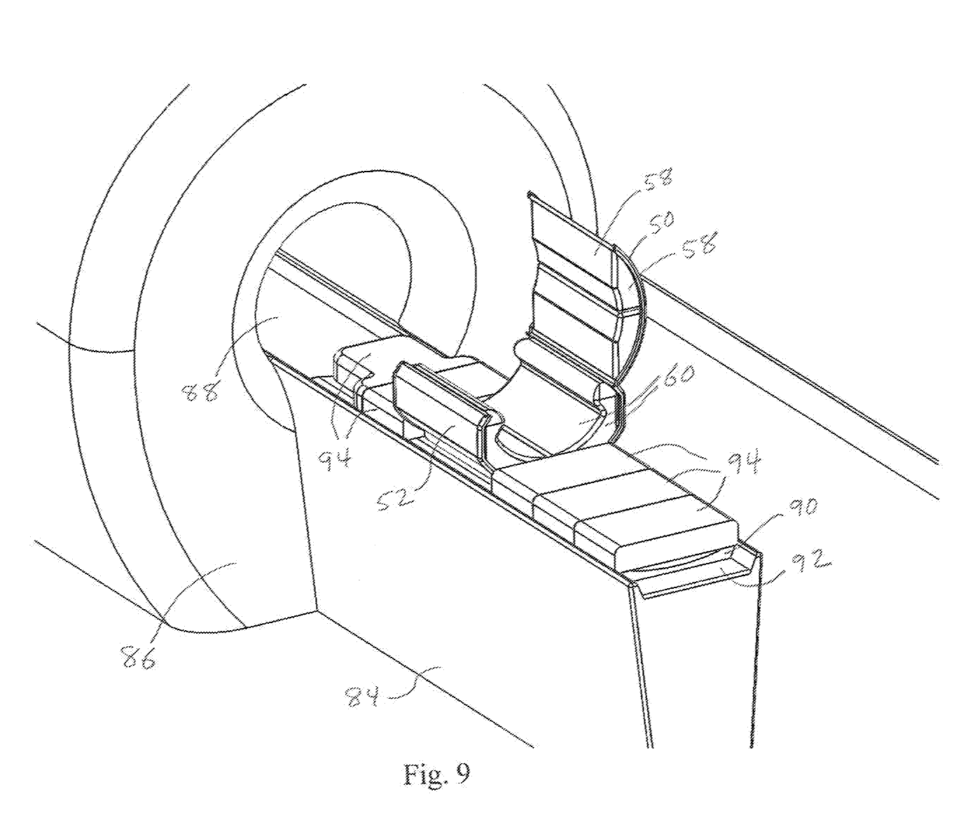

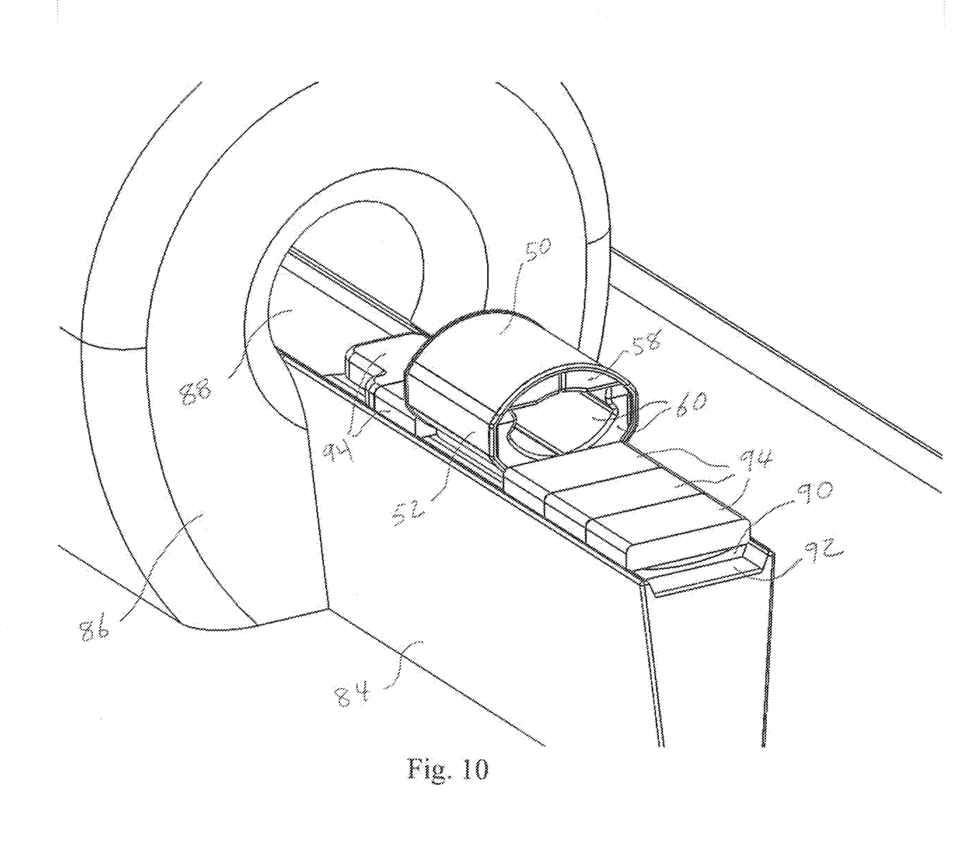

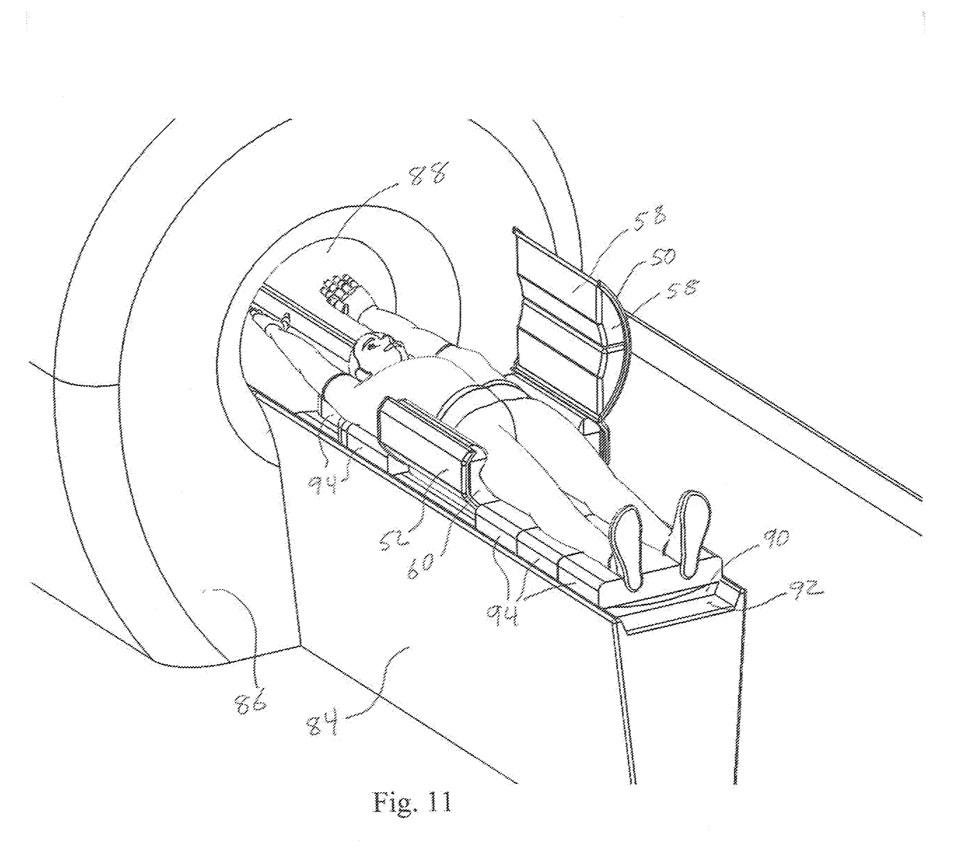

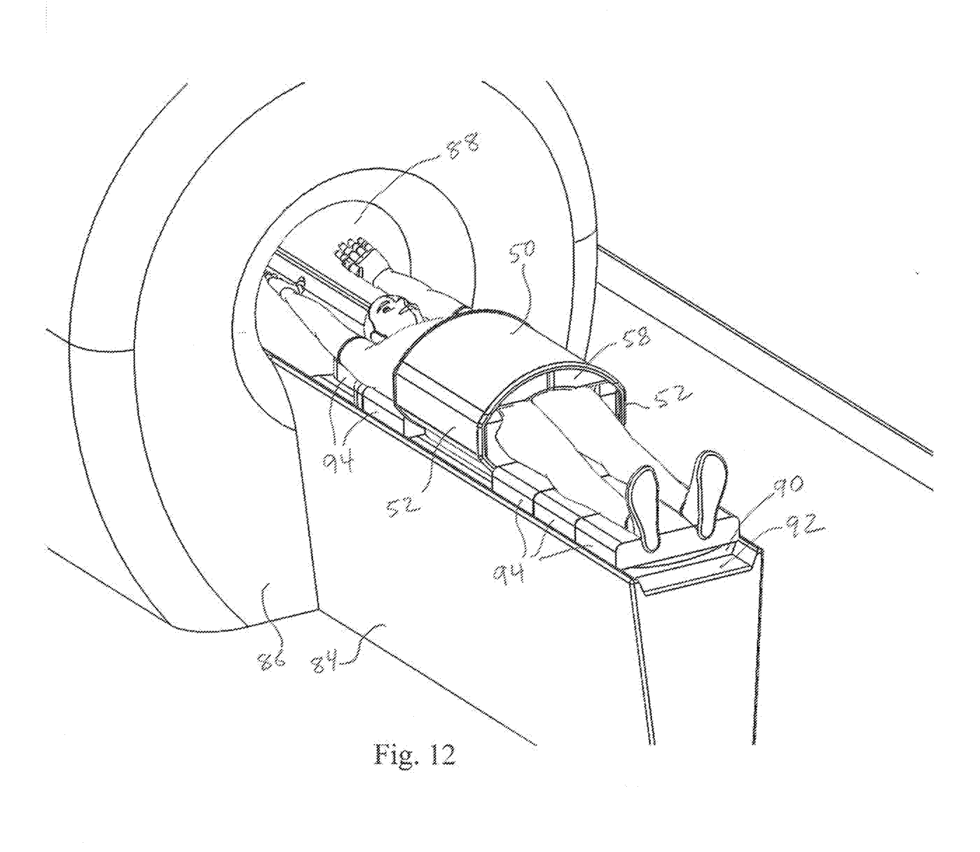

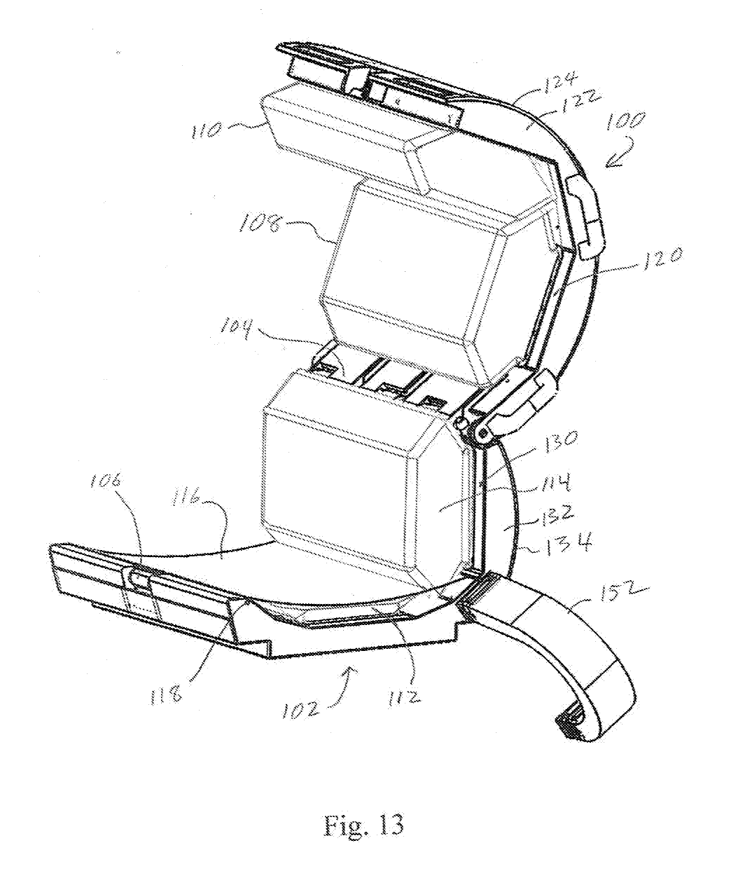

As can be seen, the EMR applicator housing of the invention does not include a patient support which extends outside of the EMR applicator shell. FIG. 9 shows the example illustrated EMR applicator in open position on a base 84, while FIG. 10 shows the EMR applicator in closed position on base 84. As shown in FIGS. 9-12, base 84 is a supporting base extending from an MRI system 86 with opening 88 which includes a movable table 90 in a channel 92 in the top of base 84. Table 94 is the usual patient table provided for a patient to lie on for movement into and out of opening 88 in an MRI system 86 for normal MRI imaging use. As shown in FIG. 9, the EMR applicator housing of the invention is positioned on this usual patient table 90 along with a plurality of cushions or pieces of foam padding 94. To obtain a hyperthermia treatment in an MRI system, an EMR applicator housing of the invention is placed on the MRI system patient table 90 and cushions 94 are placed on the patient table 90 adjacent each end of the EMR applicator housing. The EMR applicator housing is placed in open condition as shown in FIG. 9. A patient sits on a cushion 94 at one end of the open EMR applicator housing, for example the end away from the MRI opening 88, and then rotating ninety degrees and lying down in the applicator, positions himself or herself in the lower housing shell 52 of the open EMR applicator housing as shown in FIG. 11. The pads 94 would then support the patient to the approximate center of the EMR applicator array. The lower water bolus formed by material 60 can be already filled with deionized water to provide a water bed type of support surface for the patient as he or she lies down in the lower housing shell 52. When appropriately positioned in the lower housing shell 52 (measurements of the patient's position with respect to the ends and sides of the lower housing shell of the EMR applicator housing and the height of the patient in the lower housing shell of the EMR applicator housing can be made), the upper housing shell 50 is closed over the patient, the upper shell latched in closed position, and the material 58 forming the upper bolus is filled with deionized water before proceeding to the hyperthermia treatment, see FIG. 12. In this position with the EMR applicator housing closed, the position of the patient within the closed EMR applicator housing can be examined and can be adjusted, if necessary, by adjusting the inflation of the upper and lower boluses. This can ensure that the patient is exactly positioned as desired for the treatment. The patient table 90 is then moved into the MRI system opening 88 to move the patient into the MRI system 86. Once in the MRI system, hyperthermia treatment takes place by operation of the EMR applicators in the EMR applicator housing and the tissue temperature during the hyperthermia treatment is monitored by the MRI system as described in referenced U.S. Pat. No. 8,170,643. It should be realized that the applicators in the applicator housing will be connected to a remote power supply and control unit so that one or more cables, such as coaxial cables, not shown, will be connected between the applicator housing and the remote power and control unit. In addition, inlet and outlet hoses, not shown, for inflating or deflating the boluses, and for circulating fluid in the boluses, if desired, may also be attached to the applicator housing when inserted into the MRI system.

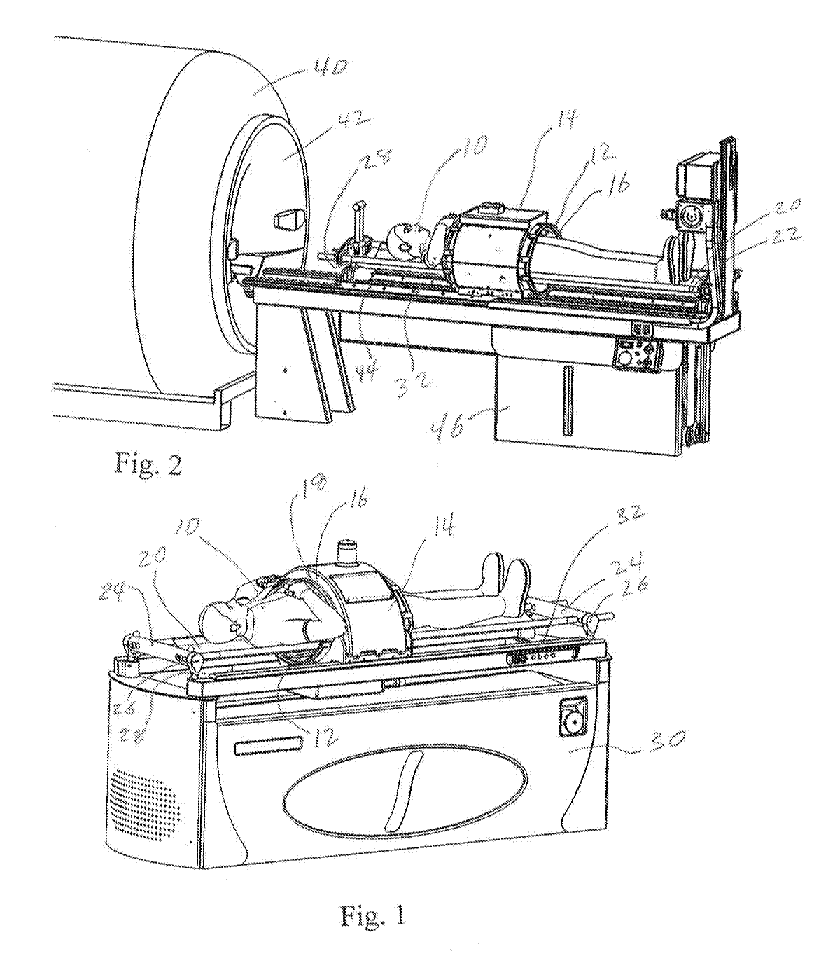

If an MRI system is not being used as part of the hyperthermia treatment, the EMR applicator housing of the invention can still be used in place of the prior art system housing such as shown in FIG. 1, by replacing the applicator housing 14, the fabric sling 20 and fabric sling supporting and adjusting structure (side support tubes 22, end support tubes 24, vertical corner supports 28, and the corner support height adjustment system) with the EMR applicator housing of the invention and the cushions or padding 94 adjacent the ends of the EMR applicator housing placed on the top surface of the system base 30. This will provide a simplified patient support system in comparison with the patient support system shown in FIG. 1.

By using support pads and the clam shell EMR applicator housing of the invention, the fit of the structure to provide the hyperthermia treatments will make it possible to adapt these structures to fit onto various standard patient support structures.

The various connectors for connecting the upper housing shell to the lower housing shell when in closed position, such as indicated hinging connectors and latch connectors, can be connected so as to allow some movement of the upper housing shell with respect to the lower housing shell when in closed position, such as movement toward or away from one another, to provide additional adjustability of the patient position in the housing by inflation of or deflation of the lower bolus or boluses.

FIGS. 13-15 show an embodiment of the EMR applicator housing of the invention with the clam shell arranged to provide the split for the applicator housing lid at a bottom corner of the housing. In the embodiment of FIGS. 3-8, the patient is centered in the applicator, surrounded by the boluses. The lower opening provided by the split at the bottom corner of the housing permits easier entry by the patient into the housing. In addition, the embodiment of FIGS. 13-15 provides a sling patient support connected to the applicator walls to fix the back plane for the patient through the applicator housing. This provides a substantially fixed support for a patient in the applicator housing and does not rely on the inflation of the bottom bolus to adjust and fix the position of the patient within the applicator housing. A more fixed location for the patient back relative to the applicator housing and to the phased array of applicators positioned in the housing provides a more consistent treatment location of the patient in the applicator housing. This is helpful for knowing a predetermined and easily achieved repeatable location of the patient relative to the phased array not only for actual patient treatment, but also for numerical pretreatment planning calculations for the phase and power amplitude needed to direct the focus to the target tissue. Past practice has been to try to center the patient's body in the center of the array. This is then assumed to be the location for the numerical treatment position. However, it is not at all easy to actually locate the patient's body in the center of the array. This is because with the prior art systems such as shown in FIGS. 1 and 2, the patient has been treated while supported on a sling stretcher which bends with the patient weight. Also, the patient's body is not easily viewed for its position within the array because the applicator outer covers and shell obstruct the view of the patient. While the embodiments of the applicator housing shown in FIGS. 3-8 alleviate this problem to some extent by directly supporting the portion of the patent's body in the applicator housing with the bottom bolus where the bolus inflation can be controlled, the patient's body still is not easily viewed for its position within the array because the applicator housing's outer covers and shell still obstruct the view of the patient within the applicator housing. With a sling support for the portion of the patient's body within the applicator housing, the portion of the patient's body within the housing is positioned at a common fixed position which provides for a standard predetermined position of the body to input into the numerical pretreatment modeling and for preplanning of the proper parameters needed to focus the heating to the target tissue.

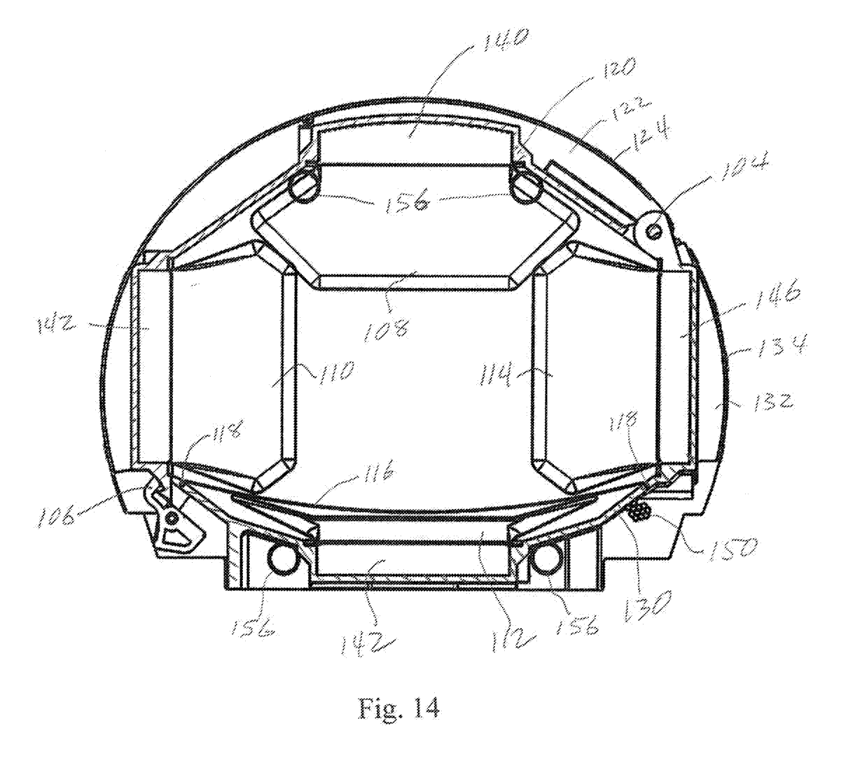

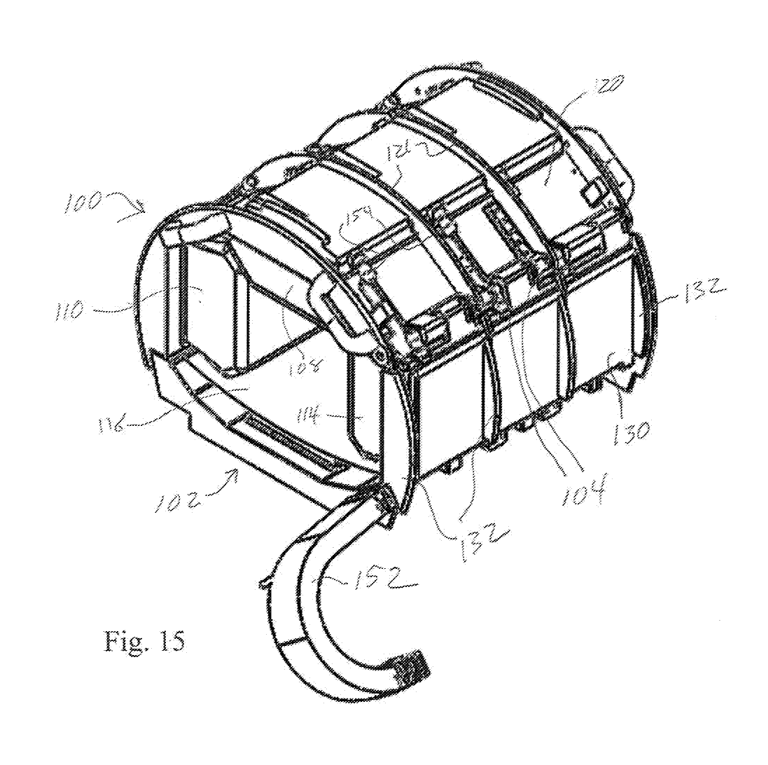

Referring to FIGS. 13-15, an upper housing section 100 is hingedly secured to a lower housing section 102 by hinge connectors 104. The housing sections can be moved between an open condition as shown in FIG. 13 and a closed condition as shown in FIGS. 14 and 15. A connector 106 can latch the upper and lower housing sections together in closed condition. Upper housing section 102 has a top bolus 108 and a side bolus 110 extending therefrom and lower housing section 102 has a bottom bolus 112 and an opposite side bolus 114 extending therefrom. A sling 116, such as a fabric or plastic sling, extends across the bottom of lower housing section 102 above bottom bolus 112, with opposite sides of sling 116 secured to lower housing section 102 in any suitable manner. For example, the sling material may be looped along opposite sides with a small rod running through respective loops. These loops, enlarged by the rods running through them, and indicated generally by reference number 118, are held in place in respective channels in the applicator frame.

This applicator housing is used in the same manner as is the applicator housing embodiment of FIGS. 3-8 and would replace the applicator housing of FIGS. 3-8 as shown in FIGS. 9-12. Similarly to the description of FIG. 9, if the applicator of FIGS. 13-15 is used in FIGS. 9-12, as previously described, a patient sits on a cushion 94 at one end of the open EMR applicator housing, for example the end away from the MRI opening 88, and then rotating ninety degrees and lying down in the applicator, positions himself or herself on the sling 116 in the lower housing shell 102 of the open EMR applicator housing as shown in FIG. 11. The pads 94 would then support the patient at the approximate level of the sling 116 in the applicator housing of FIGS. 13-15. The lower water bolus 112 can be already filled with deionized water to abut the bottom surface of the sling 116, but, the patient is supported in the applicator housing by sling 116, not by the bottom bolus 112. When appropriately positioned in the lower housing shell 102, the upper housing shell 100 is closed over the patient, the upper shell latched in closed position, and the upper bolus 108 and the side boluses 110 and 114 are filled with deionized water before proceeding to the hyperthermia treatment, similarly as shown in FIG. 12.

In the embodiment of FIGS. 13-15, the housing sections are constructed with structural ribs, walls, and covers. Thus, upper housing section 100 includes inner wall 120, ribs 122, and cover 124. Lower housing section 102 includes inner wall 130, ribs 132, and cover 134. The inner walls of the upper and lower housing sections form inner surfaces which correspond to the inner upper concave surface 51 and inner lower concave surface 53 of the housing sections of the embodiments of FIGS. 3-8. However, the inner wall 120 of the upper housing section 100 includes top pockets 140 and side pockets 142 which form part of the top and side boluses, extend the depth of the boluses, and which provide mounting surfaces for the actual EMR applicators, such as dipole antennas, not shown, in order to separate the antennas from the patient surface the minimum distance required. Similarly, inner wall 130 of the lower housing section 102 includes bottom pockets 144 and opposite side pockets 146. These also provide mounting surfaces for the actual EMR applicators, such as dipole antennas, not shown, in order to separate the antennas from the patient surface the minimum distance required. Again electrical cables will connect the EMR applicators to a source of EMR energy outside of the applicator housing. Twelve of these cables, which, for example would each connect to a pair of dipole antennas, are shown schematically in the lower housing section by reference number 150, FIG. 14. These cables will extend from the applicator housing to connect with the source of EMR energy outside of the applicator housing. These cables can enter and leave the applicator housing through a protective sheath 152, such as a cable chain. These cables have taken the form of coaxial cables in prior art hyperthermia systems. However, it has been found that with the applicator housings of the invention, it can be advantageous to use twinax cables instead of the usual coaxial cables. The use of the twinax cable instead of coaxial cable provides a balanced feeding transmission line for the dipole antenna which is a balanced antenna type. Using the twinax cable has the advantage of having a neutral grounded shield over the two inner RF active wires which reduces the RF fields on the outer portion of the outer shield. That reduces the cross coupling of RF between the cables that are connected to the antenna. This is good because RF power that cross couples from the outside of one cable to another can alter the distribution of the heating pattern by allowing cross coupled RF power to be included in the transmitted RF power. In doing so the cross coupled RF power would not be controlled in phase or power level by the amplifier system leading to a degraded heating pattern focus.

As with the applicator housing embodiments of FIGS. 3-8, a dielectric fluid has to be provided to the boluses. This requires connection of tubing between the boluses of the applicator housings and a source of dielectric fluid outside the applicator housings. As shown in FIG. 15, fluid tubing connectors 154 are shown connecting to top pocket 140 through upper housing section inner wall 120. Fluid supply tubing connects to the fluid tubing connectors and extends from the applicator housing to connect with the source of dielectric fluid outside of the applicator housing. The fluid supply tubing can enter and leave the applicator housing with the electrical cables through the same protective sheath 152 as the electrical cables.

The applicator housings of FIGS. 13-15 may also include the gel tubes 156 as described for gel tubes 89 of the embodiments of FIGS. 3-8. Here, however, the gel tubes 156 in the lower housing section are located outside of the boluses. However, they provide similar information for compensation of the temperature measurement taken by an MRI system.

Whereas the invention is here illustrated and described with reference to an embodiment thereof presently contemplated as the best mode of carrying out the invention in actual practice, it is to be understood that various changes may be made in adapting the invention to different embodiments without departing from the broader inventive concepts disclosed herein and comprehended by the claims that follow:

1. An electromagnetic energy applicator housing for positioning an array of electromagnetic energy applicators around an opening adapted to receive a portion of a patient body having tissue therein in need of hyperthermia treatment, wherein the electromagnetic energy applicator housing can move between an open condition to directly receive a portion of the portion of the patient body having the tissue therein in need of hyperthermia treatment and a closed condition for treatment, comprising:
a lower housing shell forming an inner lower concave surface and having opposite lower housing shell sides and opposite lower housing shell ends;
a lower bolus extending from the inner lower concave surface of the lower housing shell and adapted to be filled with a dielectric fluid, said lower bolus having a lower bolus surface spaced from the inner lower concave surface when filled with a dielectric fluid and adapted to receive a portion of the portion of the patient body having tissue therein in need of hyperthermia treatment when the portion of the patient body having tissue therein in need of hyperthermia treatment is to be received in the electromagnetic energy applicator housing;
an upper housing shell forming an inner upper concave surface and having opposite upper housing shell sides and opposite upper housing shell ends;
an upper bolus extending from the inner upper concave surface of the upper housing shell and adapted to be filled with a dielectric fluid;
connectors adapted to connect sides of the upper housing shell to sides of the lower housing shell in a manner that the inner upper concave surface faces the inner lower concave surface to create an opening between the upper housing shell and the lower housing shell extending between opposite ends of the upper and lower housing shells when the upper housing shell and lower housing shell are connected creating a closed condition of the upper and lower housing shells;
a plurality of electromagnetic energy applicators positioned on the inner lower concave surface of the lower housing shell and the inner upper concave surface of the upper housing shell so as to create, when the housing shells are in closed condition, at least one ring of a plurality of electromagnetic energy applicators around the opening adapted to receive a portion of a patient body for hyperthermia treatment;
means for connecting the plurality of electromagnetic energy applicators to a source of electromagnetic energy;
means for connecting the lower bolus to a source of fluid to fill the lower bolus with dielectric fluid; and
means for connecting the upper bolus to a source of fluid to fill the upper bolus with dielectric fluid.

2. An electromagnetic energy applicator housing according to claim 1, wherein the connectors adapted to connect sides of the upper housing shell to sides of the lower housing shell include a hinged connector connecting one of the opposite sides of the lower housing shell to one of the opposite sides of the upper housing shell so that the upper housing shell and lower housing shell can be rotated with respect to one another between the closed condition and the open condition.

3. An electromagnetic energy applicator housing according to claim 2, wherein the connectors adapted to connect sides of the upper housing shell to sides of the lower housing shell lock the upper housing shell to the lower housing shell to prevent relative movement of the housing shells when in closed condition.

4. An electromagnetic energy applicator housing according to claim 2, wherein the connectors adapted to connect sides of the upper housing shell to sides of the lower housing shell allow controlled movement of the respective shells toward and away from one another when in closed condition.

5. An electromagnetic energy applicator housing according to claim 2, wherein the upper bolus and the lower bolus are each two separate boluses.

6. An electromagnetic energy applicator housing according to claim 5, wherein low dielectric sector separators are positioned between adjacent bolus sides.

10. A patient support system for supporting a patient for hyperthermia treatment, comprising:
a patient support surface;
an electromagnetic energy applicator housing positioned on the patient support surface and positioning an array of electromagnetic energy applicators around an opening adapted to receive a portion of a patient body having tissue therein in need of hyperthermia treatment, wherein the electromagnetic energy applicator housing can move between an open condition to directly receive a portion of the portion of the patient body having the tissue therein in need of hyperthermia treatment and a closed condition for treatment, wherein the electromagnetic applicator housing comprises:
a lower housing shell forming an inner lower concave surface and having opposite lower housing shell sides and opposite lower housing shell ends;
a lower bolus extending from the inner lower concave surface of the lower housing shell and adapted to be filled with a dielectric fluid, said lower bolus having a lower bolus surface spaced from the inner lower concave surface when filled with a dielectric fluid and adapted to receive a portion of the portion of the patient body having tissue therein in need of hyperthermia treatment when the portion of the patient body having tissue therein in need of hyperthermia treatment is to be received in the electromagnetic energy applicator housing;
an upper housing shell forming an inner upper concave surface and having opposite upper housing shell sides and opposite upper housing shell ends;
an upper bolus extending from the inner upper concave surface of the upper housing shell and adapted to be filled with a dielectric fluid;
connectors adapted to connect sides of the upper housing shell to sides of the lower housing shell in a manner that the inner upper concave surface faces the inner lower concave surface to create an opening between the upper housing shell and the lower housing shell extending between opposite ends of the upper and lower housing shells when the upper housing shell and lower housing shell are connected creating a closed condition of the upper and lower housing shells;
a plurality of electromagnetic energy applicators positioned on the inner lower concave surface of the lower housing shell and the inner upper concave surface of the upper housing shell so as to create, when the housing shells are in closed condition, at least one ring of a plurality of electromagnetic energy applicators around the opening adapted to receive a portion of a patient body for hyperthermia treatment;
means for connecting the plurality of electromagnetic energy applicators to a source of electromagnetic energy;
means for connecting the lower bolus to a source of fluid to fill the lower bolus with dielectric fluid; and
means for connecting the upper bolus to a source of fluid to fill the upper bolus with dielectric fluid;
a pad positioned on the support surface adjacent one end of the electromagnetic energy applicator housing adapted to support a portion of the patient body extending from the one end of the electromagnetic energy applicator body in a desired position with respect to the one end of the electromagnetic energy applicator housing; and
a pad positioned on the support surface adjacent the opposite end of the electromagnetic energy applicator housing adapted to support a portion of the patient body extending from the opposite end of the electromagnetic energy applicator body in a desired position with respect to the opposite end of the electromagnetic energy applicator housing.
